(19) United States
(12) Patent Application Publication
(10) Pub. No.: US 2007/0055210 A1
(43) Pub. Date: Mar. 8, 2007

(54) SANITARY NAPKIN

(75) Inventor: Wan-Sheng Kao, Taipei (TW)

Correspondence Address: NIKOLAI & MERSEREAU, P.A., 900 SECOND AVENUE SOUTH, SUITE 820, MINNEAPOLIS, MN, 55402, US

(73) Assignee: MAMA HAPPILY DRESS FASCH LTD., Taipei (TW)

(21) Appl. No.: 11/307,409

(22) Filed: Feb. 6, 2006

Related U.S. Application Data
(63) Continuation-in-part of application No. 11/162,313, filed on Sep. 6, 2005, now abandoned.

(51) Int. Cl. A61F 13/15

(52) U.S. Cl. 604/361; 604/385.101; 604/367

(57) ABSTRACT

A sanitary napkin has a top layer, an absorbent layer and a bottom layer with a sensor layer. The absorbent layer has a hollow portion covered with a silk-made cover layer and has multiple holes defined in the cover layer, wherein some silk cloth filled inside the hollow portion. By having the hollow portion with silk cloth, the menstrual blood is drained quickly and absorbed by the absorbent layer to keep the sanitary napkin dry at the top layer and to avoid toxic vapor. Moreover, the holes on the cover layer provide a ventilating efficiency and the silk is cool to be contact so that muggy feeling is efficiently reduced. The sensor at the bottom layer conveniently indicates the changing occasion to the user.

This is a continuation-in-part (CIP) of U.S. patent application Ser. No. 11/162,313, filed on Sep. 6, 2005, which is incorporated herein by reference.

### BACKGROUND OF THE INVENTION

1. Field of the Invention

The present invention relates to a sanitary napkin, and more particularly to a sanitary napkin that makes the menstrual blood dispersed evenly and quickly and away from a vaginal opening so that an uncomfortable feeling and toxic vapor caused by the menstrual blood with heat are obviated to keep the sanitary napkin safe in use.

2. Description of Related Art

Females at the age of puberty have sexual developments such as uterus maturation that causes menses at normally monthly intervals. During menstruation, uterine intima is stimulated by estrogen, gets thick and then degenerates to separate from the uterus and to be discharged via the vaginal opening with menstrual blood. Menses is a particular sexual feature to the females and causes troubles to the females since ancient age.

According to historical papers, women in age of cave dwelling use sponges or hay to absorb the menstrual blood. Some women in ancient Egypt use softened Egyptian paper reed to make absorbent strips and some Greek women use linen wrapping around small sticks to make absorbent devices for menses treatment or take clean cotton to make pads that can be reused after washing. In China, women put an elongated clean cloth at the crotch to absorb the menstrual blood. After use, the clean cloth is washed with water and alum for reuse. Because the menses cause many troubles, women do not go on a long journey in ancient age during menstruation.

In the beginning of twentieth century, some companies manufacture napkin products. One conventional napkin product was an absorbent sheet attached on an undergarment by safety pins. Another conventional napkin product was a menstrual pad composed of the absorbent sheet with multiple belts to tie on the waist of a user. However, the menstrual pad is still inconvenient in use. Until 1921, Kimberly Clark Company manufactured a first disposable sanitary napkin that was the original model of all various sanitary napkin products using now. Thereby, women are not bothered by the inconvenience of the menstrual problems anymore.

A conventional napkin has a top layer made of soft cotton textile, an absorbent core made of board pulp embedded inside the conventional napkin and an anti-leakage membrane attached between the top layer and the absorbent core. The conventional napkin further has a glue layer formed at a bottom and two wings extending from sides to keep the napkin from sliding on the undergarment so that the menstrual blood can be completely absorbed by the absorbent core.

Although the conventional napkin above provides a solution for menstrual problems, some drawbacks of the conventional napkin still exist. Because the menstrual blood flows randomly, the female user feels uncomfortable about directly contacting the warm menstrual blood especially when the menstrual blood is plenty. To this drawback, manufacturers developed another conventional napkin that has the absorbent core shaped into a central bulge and two side low portions in accordance with the crutch to make the menstrual blood been absorbed well when the menstrual blood just comes out of the vaginal opening. However, the menstrual blood is warm and substantially gathered at the central bulge that directly abuts the crutch to cause an uncomfortable burning feeling to the female user. When the central bulge abuts tightly against the crutch, the menstrual blood can not be absorbed quickly. Moreover, heat of the menstrual blood causes trace vaporization of polyvinyl chloride monomer that is toxic, enters the vagina and causes some pathological changes at the cervix.

### SUMMARY OF THE INVENTION

One main objective of the present invention is to provide a sanitary napkin that makes the menstrual blood quickly dispersed in an absorbent material and away from the crotch to avoid uncomfortable feeling so that the menstrual blood does not cause any toxic vapor due to heating of the absorbent material.

Another main objective of the present invention is to provide a sanitary napkin that has a hollow portion for storing and a silk cloth for conducting to make the menstrual blood been absorbed by the absorbent material. By further having multiple holes in a covering layer, the sanitary napkin is ventilative to keep the crutch dry and comfortable.

Still another main objective of the present invention is to provide a sanitary napkin that has an opening defined in a bottom layer and a sensor layer partially attached at the opening to indicate a changing occasion by color changes after the sensor layer is stained with the menstrual blood.

To achieve the foregoing objective, the sanitary napkin comprises a top layer, an absorbent layer and a bottom layer laminated in sequence, wherein improvements of the sanitary napkin comprise:

a hollow portion is defined in the top layer and the absorbent layer along a central line;

a cover layer made of silk is mounted on the top layer over the hollow portion and has multiple holes defined in the cover layer; and

multiple silk masses fills the hollow portion to conduct menstrual blood to the absorbent layer.

Thereby, the sanitary napkin has excellent absorbent efficiency and always keeps dry and cool at the top layer.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a sanitary napkin in accordance with the present invention;

FIG. 2 is a cross-sectional side view of the sanitary napkin in FIG. 1; and

FIG. 3 is an operational cross-sectional side view of the sanitary napkin in FIG. 1.

### DETAILED DESCRIPTION OF THE PREFERRED EMBODIMENT

A sanitary napkin in accordance with the present invention comprises a top layer, an absorbent layer and a bottom layer with a sensor layer. The absorbent layer has a hollow portion covered with a silk-made cover layer and has multiple holes defined in the cover layer, wherein some silk cloth fills inside the hollow portion. By having the hollow portion with silk cloth, the menstrual blood is drained quickly and absorbed by the absorbent layer to keep the sanitary napkin dry at the top layer and to avoid toxic vapor. Moreover, the holes on the cover layer provide a ventilating efficiency and the silk is cool to be contacted so that muggy feeling is efficiently reduced. The sensor at the bottom layer is preferably made of indicator paper and conveniently indicates the changing occasion to the user.

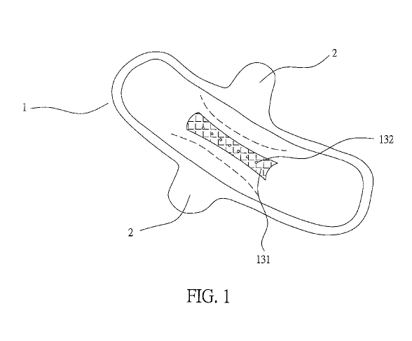

With reference to FIG. 1, a preferred embodiment of the sanitary napkin comprises a pad body 1 and two wings 2 formed at two sides of the pad body 1, wherein a cover layer 131 with multiple holes 132 is formed at a center transversal line of the pad body 1.

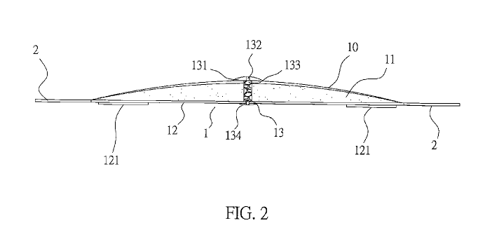

As shown in FIG. 2, the pad body 1 comprises a top layer 10, an absorbent layer 11 and a bottom layer 12. The top layer 10 is made of silk cloth that is smooth in texture and super-ventilative to make contact gentle and comfortable without muggy feeling. The absorbent 11 layer is made of 100% pure ceiba pulp or a mixture of macromolecular polymers to quickly absorb warm menstrual blood and eliminate thermal and unpleasant odors. The bottom layer 12 is made of super-thin plastic membrane having anti-leakage efficiency and has two glue strips 121 attached on an outer side of the bottom layer 12 for attaching convenience. Especially, the whole pad body 1 has an elongated hollow portion 13 defined along the center transversal line from the top layer 10 to the absorbent layer 11 inside the pad body 1 to correspond to the crutch and to facilitate the menstrual blood to disperse downward. The cover layer 131 made of silk covers an opening of the hollow portion 13 on the top layer 10 and has the multiple holes 132 defined in the cover layer 131 to make the sanitary napkin ventilative. Moreover, the hollow portion 13 is filled with irregular silk masses 133 to absorb and help the menstrual blood to disperse outward in the absorbent layer 11 so that the crutch has no pigmentations or sanitary problems caused by the damp of the menstrual blood. Additionally, the bottom layer 12 has an aperture and a sensor layer 134 partially covering the aperture. When the menstrual blood contacts the sensor layer, color of the sensor layer varies that means the sanitary napkin should be changed.

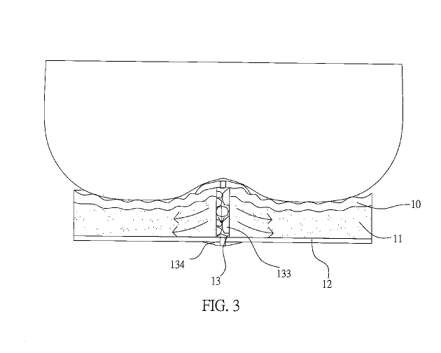

With further reference to FIG. 3, when the sanitary napkin is attached to the undergarment, the hollow portion 13 aligns to the vagina opening so that the menstrual blood quickly flows into the hollow portion 13 via the holes 132 on the cover layer 131. Meanwhile, the silk masses 133 keep the menstrual blood from reflowing back to the top layer 10 and then conduct the menstrual blood to quickly disperse to the absorbent layer 11. Thereby, the menstrual blood can be quickly absorbed by the sanitary napkin and the crutch is kept dry and clean all the time to make the user feel comfortable when the menstrual blood is plenty, the sensor layer 134 shows color changes to indicate that the sanitary napkin should be change because the absorbent layer 11 is saturated, wherein the user observes the color changes via the bottom of the panty.

According to above description, the sanitary napkin has the following advantages:

1. Because the sanitary napkin has the hollow portion 13 at the central transversal line, the menstrual blood can quickly flows into the hollow portion 13 and the silk masses 133 conduct the menstrual blood to quickly disperse to the absorbent layer 12 so that the top layer 10 is kept dry.

2. Heat of the menstrual blood is quickly reduced after dispersing to the absorbent layer 11. Therefore, chemical reaction does not happen when the heat is low and no toxic gas will be generated so that possible pathological changes are eliminated.

3. The holes 132 on the cover layer 131 provide ventilation efficiency to avoid damp so as to reduce the uneasy feeling when the female uses the sanitary napkin.

Although this invention has been described in its preferred form with a certain degree of particularity, it is understood that the present invention of the preferred form has been made only by way of example and that numerous changes in the details of construction and the combination and arrangement of parts any be resorted to without departing from the spirit and scope of the invention.

What is claimed is:

1. A sanitary napkin comprising a top layer, an absorbent layer and a bottom layer laminated in sequence, wherein improvements of the sanitary napkin comprise:
a hollow portion is defined in the top layer and the absorbent layer along a central line;
a cover layer made of silk is mounted on the top layer over the hollow portion and has multiple holes defined in the cover layer; and
multiple silk masses fills the hollow portion to conduct menstrual blood to the absorbent layer.

2. The sanitary napkin as claimed in claim 1, wherein the top layer is made of silk cloth.

3. The sanitary napkin as claimed in claim 1, wherein the absorbent layer is made of pure ceiba pulp.

4. The sanitary napkin as claimed in claim 1, wherein the absorbent layer is made of macromolecule polymer.

5. The sanitary napkin as claimed in claim 1, wherein the bottom layer has an opening defined in the bottom layer to align to the hollow portion; and
a sensor attached to the bottom layer and to partially cover the opening of the bottom layer, wherein the sensor indicates by color changes.
